(19) United States
(12) Patent Application Publication
(10) Pub. No.: US 2003/0175148 A1
(43) Pub. Date: Sep. 18, 2003

(54) Methods, devices, and articles for controlling the release of volatile materials

(75) Inventors: Frank Andrej Kvietok, Cincinnati, OH (US); Charles Michael Hardy, Norwood, OH (US); Brice Daniel Westring, Loveland, OH (US); Rachel T. Murdell, Cleves, OH (US); Eric Richard Bartsch, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/098,581

(22) Filed: Mar. 15, 2002

(51) Int. Cl. A61L 9/00; G01D 11/26; A62B 7/08

(52) U.S. Cl. 422/5; 422/119; 422/124; 422/305; 422/306; 422/307

(57) ABSTRACT

Methods, devices, and articles for controlling the release of volatile materials are disclosed, including, but not limited to scented materials. The method can be applied to a wide variety of different types of emitting devices.

### FIELD OF THE INVENTION

The present invention relates to methods, devices, and articles for controlling the release of volatile materials. Volatile materials may include, but are not limited to scented materials.

### BACKGROUND OF THE INVENTION

U.S. Pat. No. 4,629,604 issued to Spector is directed to a multi-aroma cartridge player. In a preferred embodiment, the aroma player is integrated with a video tape machine so that one can provide visual and sound entertainment in conjunction with a synchronized aroma presentation. This reference recognizes that some aromas may be more “pungent” than others, and provides that each heater in the player can be separately adjusted to provide an appropriate level of scented material.

U.S. Pat. No. 4,695,434 issued to Spector is directed to an aroma-generating unit that is adapted to discharge into the atmosphere bursts of aromatic vapor, with the non-aromatic intervals between the bursts having a duration sufficient to avoid desensitizing the olfactory response of those exposed to the unit.

Individual volatile materials differ from other volatile materials in a number of respects. Such materials differ from each other in characteristics that include, but are not limited to their volatility, their intensity when released, and their longevity after release.

The output of devices for emitting volatile materials are affected by, and in many cases adversely affected by, the characteristics of the volatile materials that they release. For example, if the volatility of the volatile material is too high, the volatile material can be used up too quickly. In the case of scented materials, if the intensity of the volatile material is too low, it may not be as noticeable as desired. If the intensity of a scented material is too high, it may become overpowering. In the case of scented materials, if the longevity is too short, or too long, the user may not obtain the desired “scent experience”. If there is an interest in releasing multiple volatile materials with a single device, controlling the output of the device is greatly complicated by the differences between different volatile materials.

Thus, there is a need to provide improved methods for controlling the release of volatile materials, such as scented materials.

### SUMMARY OF THE INVENTION

The present invention relates to methods, devices, and articles for dispensing and controlling the release of volatile materials, including, but not limited to scented materials.

The methods of the present invention apply to a wide variety of different types of emitting devices. The devices can range from simple passive emitting articles, such as baking soda in a box to more complex devices capable of emitting multiple volatile materials. The devices can be controlled by the user, or they can be controlled automatically. In some embodiments, a device can emit volatile materials from an article (which article may include, but is not limited to fill or refill units, cartridges, or other articles). In such embodiments, the article can be provided with a mechanism for communicating information between the article and the device that controls the release of the volatile materials from the article. Communication with the user is also possible.

In one non-limiting embodiment, for example, the information communicated to the device by the article and/or the user may be volatile material-specific. There may also be one or more separate inputs that are not volatile material-specific (such as those related to user-preferred intensity, duration, room size, or other variables related to the use of the volatile material) that could be set by the user. These two types of input can be used in conjunction to control the application of the volatilization energy and, thus, the volatilization of the volatile material from the article.

There are also a non-limiting number of possible embodiments of the devices and articles that can carry out the methods described herein. In some embodiments, each different volatile material can be heated to a different temperature. In other non-limiting embodiments, the method can be carried out by placing films of different porosity between the volatile material and the atmosphere. In other non-limiting embodiments, the method can be carried out by providing a spacer (or some other mechanism) that adjusts the distance between the volatile material and a heater. In other, more complex embodiments, the article can convey specific information about one or more volatile materials to a device that releases the volatile materials, which instructs the device how to adjust for a particular volatile material or group of volatile materials. For example, in the case of scented materials, the device can adjust the application of energy to generate a suitable scent intensity and/or duration for the particular scented material(s). The adjustment of the device can, for example, take into account the fact that some scented materials remain in the air longer than others, and adjust the duration of application of energy to such material, such as by reducing the same, to reflect this. Numerous other embodiments are possible.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a schematic side view of one non-limiting embodiment of a device for carrying out the method of the present invention.

FIG. 2 is a schematic side view of another non-limiting embodiment of a device that can be used to carry out the method of the present invention.

FIG. 3 is a schematic side view of a non-limiting arrangement of a container for a volatile material.

FIG. 4 is a schematic top view of an embodiment of a cover for the volatile material container shown in FIG. 3 which can be used with volatile materials having a relatively high volatility.

FIG. 5 is a schematic top view of an embodiment of a cover for the volatile material container shown in FIG. 3 which can be used with volatile materials having a lower volatility.

FIG. 6 is an alternative schematic top view of an alternative cover for the volatile material container shown in FIG. 3.

FIG. 7 is an enlarged view of the portion of the cover shown in FIG. 6, which could be suitable for use with volatile materials having a relatively high volatility.

FIG. 8 is an enlarged view of the portion of the cover shown in FIG. 6, which could be suitable for use with volatile materials having a lower volatility.

FIG. 9 is a schematic fragmented side view of a fill or refill container and a portion of a device in which there is a spacer between the volatile material and a source of heat.

FIG. 10 is a top view of one embodiment of a fill or refill container.

FIG. 11 is a fragmented cross-sectional view of a fill or refill container and a portion of a device wherein the view through the container is taken along line 11-11 of FIG. 10.

FIG. 12 is a fragmented view similar to FIG. 11 which shows a container having a bottom portion that is thicker than the embodiment shown in FIG. 11.

FIG. 13 is a fragmented view similar to FIG. 11 which shows a container having additional material located between the volatile material and the source of heat.

FIG. 14 is a fragmented view similar to FIG. 11 which shows a container having additional material located between at least part of the volatile material and the source of heat.

FIG. 15 is a schematic cross-sectional view from the side of refill having positioning guides thereon and a mating refill receptacle in a device.

FIG. 16 is a schematic cross-sectional view from the side of a refill container having multiple positions for inserting a volatile material.

FIG. 17 is a perspective view of a fill or refill unit that comprises a container having a wick for dispensing the volatile material.

FIG. 18 is a top view of the unit shown in FIG. 17.

FIG. 19 is a schematic cross-sectional view taken along line 19-19 of FIG. 18 of a wick that is positioned between the tapered heating elements of a heater.

FIG. 20 is a schematic cross-sectional view similar to that of FIG. 18 of a wick that is at a lower position between the tapered heating elements of a heater.

FIG. 21 is a schematic cross-sectional view from the side of a refill assembly having a ramp thereon for adjusting the control arm that holds an insulating collar above a wick extending from a container of volatile material.

FIG. 22 is a schematic side view of a volatile material-containing article having conductive element in one of four possible locations thereon.

FIG. 23 is a partially fragmented cross-sectional view of a fill or refill unit similar to that shown in FIG. 17, also taken along line 19-19, which comprises a resistor.

FIG. 24 is an enlarged view of the portion of the unit shown in FIG. 23 containing the resistor and having threaded contacts thereon.

FIG. 25 is an enlarged view of a portion of the unit shown in FIG. 23 which shows an alternative arrangment of contacts.

FIG. 26 is a perspective view of one non-limiting embodiment of a system suitable for emitting multiple volatile materials.

FIG. 27 is a perspective view of a cartridge suitable for use with the device shown in FIG. 26 which contains contacts for communication with the device.

FIG. 28 is a perspective view of a cartridge suitable for use with the device shown in FIG. 26 which contains a conductive label for communication with the device.

FIG. 29 is a perspective view of a cartridge suitable for use with the device shown in FIG. 26 which contains a bar code label for communication with the device.

FIG. 30 is a perspective view of a cartridge suitable for use with the device shown in FIG. 26 which contains a plurality of holes for communication with the device.

FIG. 31 is a perspective view of a cartridge suitable for use with the device shown in FIG. 26 which contains a radio frequency tag for communication with the device.

FIG. 32 is a top plan view of a cartridge suitable for use with the device shown in FIG. 26 which contains a lifetime indicator for the cartridge.

FIG. 33 is a perspective view of the cartridge shown in FIG. 32 having a portion of the cartridge cut away to show the lifetime indicator.

FIG. 34 is a flow chart that shows the steps in one embodiment of an overall process for controlling the emission of volatile materials.

### DETAILED DESCRIPTION OF THE INVENTION

The present invention relates to methods, devices, and articles for emitting (or dispensing) and controlling the release of volatile materials, including, but not limited to scented materials. The volatile materials, such as scents or aromas, can be supplied to the outside environment, or to various facilities, which include but are not limited to rooms, houses, hospitals, offices, theaters, buildings, and the like, or into various vehicles such as trains, subways, automobiles, airplanes and the like.

There are numerous, non-limiting embodiments of the invention. Several non-limiting embodiments are described herein, as are steps in the methods and several components of systems, each of which may constitute an invention either in their own right or combined, in any manner, with any other steps and/or components described herein. All embodiments, even if they are only described as being “embodiments” of the invention, are intended to be non-limiting (that is, there may be other embodiments in addition to these), unless they are expressly described herein as limiting the scope of the invention.

The term “volatile materials” as used herein, refers to a material or a discrete unit comprised of multiple materials that is vaporizable, or comprises a material that is vaporizable. The terms “volatile materials”, “aroma”, and “scents”, as used herein, include, but are not limited to pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile materials including, but not limited to perfumes, aromatic materials, and scented materials, will often be comprised of one or more volatile materials (which may form a unique and/or discrete unit comprised of a collection of volatile materials). The volatile materials of interest herein can be in any suitable form including, but not limited to: solids, liquids, gels, encapsulates, wicks, and carrier materials, such as porous materials impregnated with or containing the volatile material, and combinations thereof.

The term “volatile material-specific”, as used herein, refers to at least one physical and/or chemical property of a volatile material or a discrete unit comprising a mixture of volatile materials. Examples of the physical and chemical properties of volatile materials are provided later in this specification. It should be understood that the volatile materials (including discrete units comprising mixtures of volatile materials) can be grouped into common groups (e.g., those that are volatilized at relatively low heat, or relatively high heat). In such cases, volatile material-specific information may either be provided for individual volatile materials (or for discrete units comprised of mixtures of multiple volatile materials), or it may be provided for one or more groups of individual volatile materials (or for discrete units comprised of mixtures of multiple volatile materials) that have some related or similar property.

### Devices and Articles for Emitting Volatile Material

The method described herein applies to a wide variety of different types of emitting devices and articles for emitting volatile materials. The emitting device or article can be any device or article that is capable of emitting volatile materials.

The devices and articles can range from simple passive emitting articles, such as baking soda in a box to more complex devices capable of emitting multiple volatile materials. Such articles and devices include, but are not limited to: baking soda in a box; devices that are of a type that has a cover that is manipulated to expose the volatile material; plug in devices; and devices capable of emitting multiple volatile materials. The articles and devices can be controlled by the user, or they can be controlled automatically. The devices and articles can utilize energy to emit the volatile materials. This can be ambient energy (such as convection moving air past the device or article), or energy can be supplied to the device or article.

The devices and articles may be in any suitable configuration. FIG. 1 is a schematic representation showing some of the features an emitting or dispensing device (or simply “device”) 20 can have. The device 20 may comprise a container 22 for the volatile material 24. The device 20 has at least one opening 26 for the release of the volatile material 24 into the air. The device 20 may contain a component for activating the volatile material from its “resting” state to an activated state. Such a component may include, but is not limited to a component that volatilizes or heats the volatile material 24, such as a heater 28. The dispensing device 20 may also contain a component, such as a fan 30, for diffusing or transporting the volatile material 24 into the environment or atmosphere. The device 20 may also comprise an energy source 32. The energy source 32 may use any suitable type of energy, including but not limited to: convection, solar energy, sonic energy, ultrasonic energy, thermal energy, pressure release (such as from a pump, or an aerosol can), and electrical energy. The device 20 may also have controls such as those designated generally by reference number 34. These controls 34 may include, but are not limited to an intensity control 36, and controls 38 for the heater, the fan, and the time for emitting the volatile material. The device 20 may also include a volatilization control component 40 that controls the volatilization or release of the volatile material 24.

The device 20 may contain the volatile material, or it may operate in conjunction with a separate article of manufacture that is used in association with the device as an emission system (or simply “a system”). In this latter case, the article of manufacture may contain the volatile material and this article of manufacture can be placed in the device, on the device, or otherwise be associated with the device 20. If a separate article of manufacture is used, the volatile material-containing article of manufacture may be in any suitable form. The volatile material-containing article of manufacture may be in any suitable configuration including, but not limited to in the configuration of a disk, a cartridge, or a structure of any other configuration containing volatile material(s) including but not limited to structures comprising fills or refill units for articles and/or devices. In the case of fill and refill units for articles that emit volatile materials, the fill or refill units will be considered to be volatile material-containing articles of manufacture. The article that emits volatile materials, in such a case, will be considered to be a device (or “emitter” or “diffuser”) even though it may be a relatively simple article or device, and may not have any moving parts.

The volatilization control component 40 is any component (or components) that controls the ability to volatilize the volatile material. This can include, but is not limited to one or more of the following: when the volatile material volatilizes; the rate of volatilization; and the ability to volatilize. The volatilization control component 40 can be any type of component(s) suitable for any of these purposes. The volatilization control component 40 can operate by physical, mechanical, electrical, and/or electronic mechanisms. The volatilization control component 40 can comprise a separate component or components, or it can comprise all or part of one of the components of the system (such as the device or the volatile material-containing article). The volatilization control component 40 can comprise part of the controls associated with a device, or it can interact with one or more of such controls. The volatilization control component 40 can range from a relatively simple structure, such as one that performs a single function, to a complex article that performs several functions.

The emitter and/or the volatilization control component 40 can, in various embodiments, be configured so that the volatile material is emitted in any suitable manner including but not limited to: continuously, intermittently, or both (alternatively continuously and intermittently). The manner in which the emitter is controlled to emit volatile materials may be referred to herein as the “emission program” or “emission scheme”. The emission program comprises one or more emission periods during which the volatile material is emitted, and the manner or manners in which the volatile material(s) are emitted. The actual element that carries out the emission scheme can be physical, mechanical, or electrical. A non-limiting example of a mechanical element would include, but is not limited to a timer. Non-limiting examples of electrical elements include: electrical circuitry, electronic circuitry, and computer chips. In the case of computer chips, the element that carries out the emission scheme or program may be in the form of the logic that controls the energy source.

In some embodiments, the emission program (and the application of the volatilization energy) can be intermittent and can use a pulsed sequence of emissions, such as in the case of scented materials, to minimize “habituation”, or for other reasons. A pulsed sequence can divide the emission period into an integral number of timeblocks. There may be a period at the end of an emission period during which no emission (or an amount of emission below detection) may be provided and intensity levels are intentionally allowed to drop below levels of detection. In some embodiments, the emission of the volatile material can be regular, consistent, and/or continuous, such as in some prior art scent-emitting devices that are always emitting scented material.

In other embodiments, the emission of the volatile material may be irregular, or discontinuous. If desired, such as in the case of volatile material(s), such as scented materials, the emitter can deliver a non-constant objective or sensory-judged in-air concentration profile. Such non-constant concentration profiles may include features such as the introduction of random bursts of volatile material, the gradual increase or decrease in concentration through the duration of the emission, or the intentional drop in concentration below sensory limits. This latter type of emission program can differ from some known devices which are either always emitting scented material, or which pulse the emission of scented material so that there is a continuous perceived impression of a scent. In this latter case, the emission of the scented material may sometimes be below the threshold of perceived intensity. This may also differ from an aerosol in that no human interaction is needed. It can be done automatically, or by a timer.

There are numerous non-limiting embodiments of the devices, articles, and volatilization control component 40 that can carry out the methods described herein. The devices and articles can be in any suitable configuration, including configurations in current use, or they can comprise entirely new types of devices and articles. Suitable types of components that can comprise the volatilization control component 40 include: mechanisms that expose different amounts of the volatile materials to the air; heating the volatile materials to different temperatures; altering the speed of a fan that acts on the volatile materials; resistors; materials that insulate the volatile materials to differing extents; spacers for spacing the volatile materials different distances from heaters or other energy sources; computer programs, articles and/or devices that provide input to a logic circuit which controls emission energy, etc.

As noted in the Background section, the output of devices for emitting volatile materials is affected by the characteristics of the volatile materials they release. Thus, it may be desirable to use the volatilization control component to control the release of different volatile materials in different manners to accommodate these differences. This may, for example, be a way to “normalize” the emitted characteristics of different volatile materials (without changing the characteristics of the materials themselves) so that a user of the device can experience more consistent results when using different volatile materials. In order to do this, it is generally desirable to determine the specific volatilization properties or parameters of the volatile material(s) based on physical properties and/or chemical properties of volatile material(s).

The physical and/or chemical properties of the volatile material(s) can include any one or more of the following (with the manner of testing, or standard test method to determine these physical properties provided in parentheses): (i) molecular weight of the volatile material(s); (ii) the flash point of the volatile material(s) in liquid form (ASTM D56, D93); (iii) when the volatile material is combined with one or more other materials to form a volatile material-containing composition or “matrix”, the flash point of the volatile material(s)-containing matrix (ASTM D3828, D6450); (iv) dosage requirements of the liquid volatile material(s) to achieve an acceptable perceived intensity (by having sensory experts evaluate this in a controlled test); (v) the tenacity or longevity of a dose in a given area (also by using sensory experts); (vi) the volatility of the material(s) as measured on a TGA tester or similar device (ASTM E914, E1582); (vii) the volatility of the material(s) as measured by mass loss vs. time (ASTM E1131); (viii) the vapor pressure of the volatile material(s) as a liquid; and (ix) the vapor pressure of the volatile material(s)-containing matrix (the latter two can both be determined by ASTM E2071-00, E1194-01, and E1782-98).

The information on the volatilization parameters of the volatile materials can then, if desired, be stored in any suitable form on the article or other medium containing the volatile material(s). This information can then be accessed by the user of the article and/or accessed by the device without input from a user. This information can be used to provide input to a program controlling volatilization energy, or to otherwise used to optimize the volatilization of a volatile material by controlling the application of volatilization energy. The application of volatilization energy can be controlled in various ways including, but not limited to controlling: the level of volatilization energy, the duration of application of volatilization energy, and the frequency of application of volatilization energy. All of the volatilization parameters for a given volatile material need not be stored in each case. In some embodiments, for example, it may be desirable to combine and/or simplify the manner of expressing one or more of these volatilization parameters (e.g., such as classifying different volatile materials into different groups which have at least some similar volatilization characteristics, such as a group 1, group 2, etc.)

Any known mechanism or form for storing information, from the most simple (changes in topography discussed below) to complex (e.g., computer chips), can be used to store this information on the article or medium and/or the device. If the information is stored on an article or medium, it may be desirable for the information to be accessible to the device and/or the user (the latter type of information storage may, for example, be accessible by visual inspection of the article). If the article or medium contains more than one type of volatile material, information may be stored and provided separately for each volatile material contained on or in the article. Additionally or alternatively, some or all of the information may be may be stored and provided in a combined form for more than one type of volatile materials (for example, for a collection of volatile materials contained on or in the article).

In some or all of the embodiments described herein, when a volatile material-containing article is used in conjunction with an article or device for emitting the volatile material, there can be a mechanism for the communication and/or exchange of information between these two components. The mechanism for communicating information may comprise any suitable mechanism, including but not limited to electrical, mechanical, and physical mechanisms. The device and article can, in various embodiments, send to and/or receive information from the other component. The terms “communication” and “exchange” will include all such possibilities, but do not require all such possibilities. That is, “communication” and “exchange”, as used herein, can include sending information, receiving information, and both. The term “information”, as used herein, is intended to include in the broadest sense any interaction that is capable of changing a setting on an article or a device, or altering the application of energy to a volatile material by an emission device and/or the energy input that the volatile material receives. This includes physical contact, separation, insulation, electrical contact, or any other type of action that is capable of changing a setting or altering the application of energy to a volatile material by an emission device, or that changes the emission characteristics of a device or article. The exchange of information may be referred to instead as a “means for” the communication and/or exchange of information, if specifically so described in the claims, in which such means will include all the means described in this specification plus equivalents thereof. Otherwise the mechanism described herein need not be considered a “means plus function” type element.

Any appropriate type of information can be communicated or exchanged between the device and the article. Some examples of types of information that can be communicated include, but are not limited to at least one of the following: (1) volatile material-specific release or volatilization parameters (e.g., temperature to which the volatile material should be heated, etc.); (2) volatilization energy application program selection (e.g., telling or causing the device to select one of a number of settings, for example, setting numbers 1 or 2); (3) name(s) associated with volatile materials (for example, the device can read and/or display names associated with volatile materials in an article); (4) information relating to specific volatile material-containing articles (e.g., themed cartridge 1, 2, etc.); (5) information relating to the history of use of the volatile materials in the article (e.g., how much volatile material remains in the article (or a lifetime signal)); and (6) sequence of volatilization energy application programs. If the article comprises more than one volatile material, this information can be specified separately for one or more of the individual volatile materials, or it can be specified for a collection of volatile materials contained on or in the article.

In certain embodiments, where the information is communicated between an article and a device without any input from the user, this type of communication may be referred to herein as “direct” communication. In other embodiments, the article and/or the device may communicate (for example, visually or audibly) with the person using the device who can then provide an input to the device. Thus, in this latter case, it can be said that there is indirect communication between the article and device since information is first communicated to the user, and then to the device. This exchange of information need not involve a third component (such as a computer, movie track, or the like) for the communication to take place. In some systems, there may be both direct and indirect communication.

The methods, devices, and articles can provide a particular output based upon a pre-set input, or based upon inputs to be provided. The device is not required to be able to receive any type of input. In simple embodiments, the device and/or article requires no input from the user, and need involve nothing more from the user than opening the box or other package containing the device, and removing the packaging, or taking the device out of the box. This would be an example of a pre-set input. Such a device may or may not have more than one mode of emission. In other embodiments, the device and any volatile material-containing article to be used in conjunction with the device can communicate in some suitable manner so that the particular type of volatile material in the volatile material-containing article is communicated to the device, and device adjusts to generate a suitable intensity and/or duration for the particular volatile material(s).

In addition, in some embodiments, a device may have two or more modes of emission that differ in at least one parameter. These parameters include, but are not limited to: (a) the temperature to which the volatile material is heated; (b) the duration of heating, and/or if energy is applied in a pulsed manner, the duration or “width” of each pulse; (c) the intervals between active emission (e.g., heating or fan) phases; (d) the speed at which a fan that disperses the volatile material operates; (e) the duration of operation of a fan; and (f) the intervals between operation of a fan. In any of the embodiments, the heater and/or the fan can run continuously, or either the heater and/or the fan can operate intermittently during an emission cycle (such as in a pulsed manner or in a random manner).

In these or other embodiments, there may be more than one type of communication or input. For example, there may be a first communication in which the information communicated to the device by the article (e.g., automatically) and/or the user may be volatile material-specific. In addition, one or more separate second communications or inputs that are not volatile material-specific (such as those related to user-preferred intensity, duration, room size, or other variables related to the use of the volatile material) could be set by the user. These two types of input can be used in conjunction to control the application of the volatilization energy and, thus, the volatilization of the volatile material from the article. In such an embodiment, the user input may modify the volatile material-specific input, and may, but preferably does not, negate or override the volatile material-specific input.

Some non-limiting examples of these features are described below. It should be understood that any of the embodiments shown herein as containing a single volatile material or unit of volatile materials, can be adapted to include additional volatile materials, or units of volatile materials.

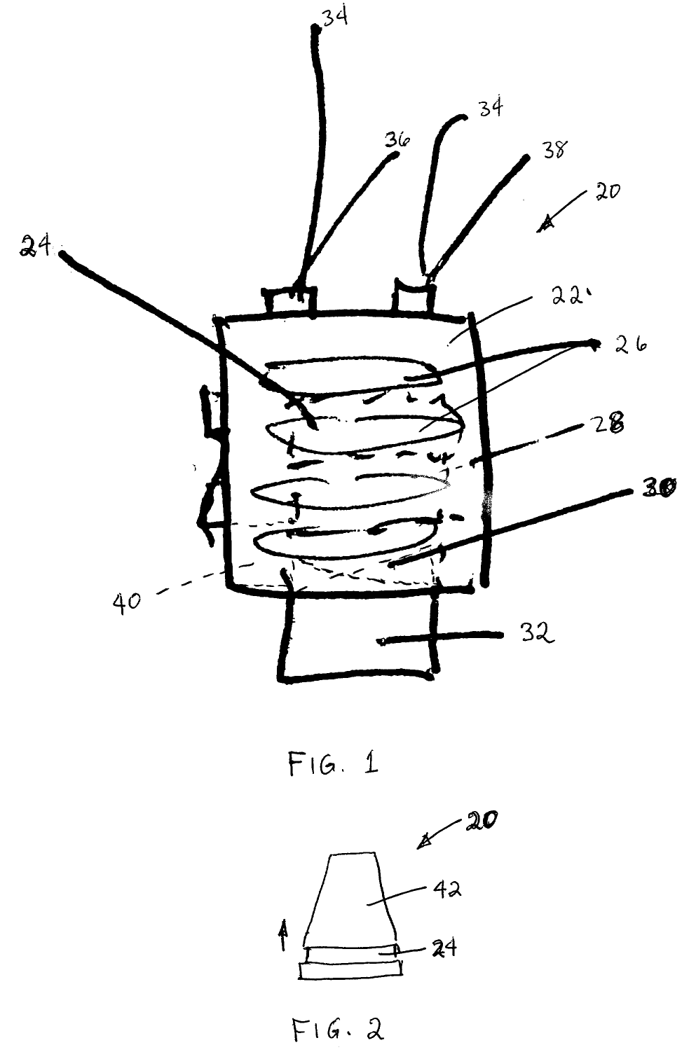

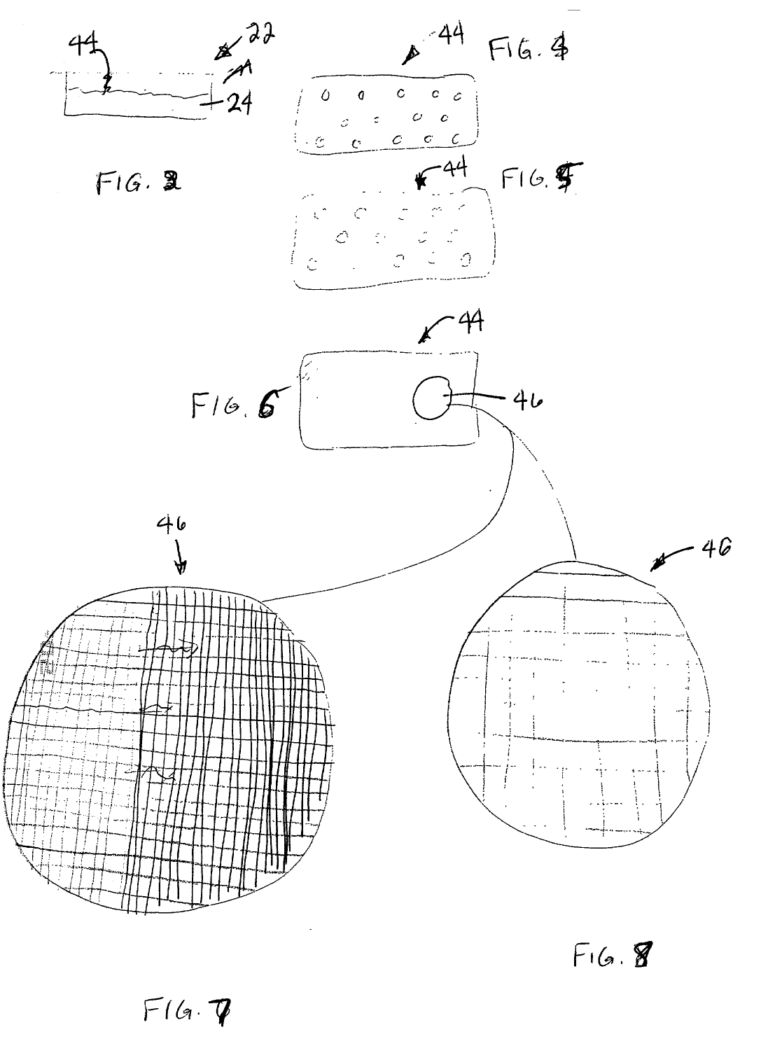

FIG. 2 shows an embodiment in which the volatilization control component 40 is systemic with a device of a particular design. FIG. 2 shows an article in which the volatile material is contained in a housing 42, and the housing 42 that can be manipulated to expose a certain amount of the surface area of the volatile material 24. The housing 42 can be manipulated in any suitable manner, including but not limited to by lifting such as in the direction of the arrow, or rotating a portion of the housing. In one version of such an embodiment, the housing 42 can lift or rotate a different amount for different scents. This could be done automatically or by the user. For various volatile materials, the housing and/or a fill or refill unit can comprise a volatilization control component that exposes a specific amount of the volatile material 24 depending on the properties of the volatile material. In the embodiment shown, the volatilization control component 40 can, for example, be a component on the housing and/or a refill for the same that limits degree to which the portion of the housing can be lifted or rotated to limit the amount of surface area of the volatile material 24 that is exposed for each particular volatile material. The present invention, however, is not limited to articles of the particular design shown in FIG. 2. Numerous other devices and articles can utilize the same principle, and may operate in a different manner including, but not limited to devices and articles having openings of other configuration, such as doors to expose various amounts of the surface area of the volatile material.

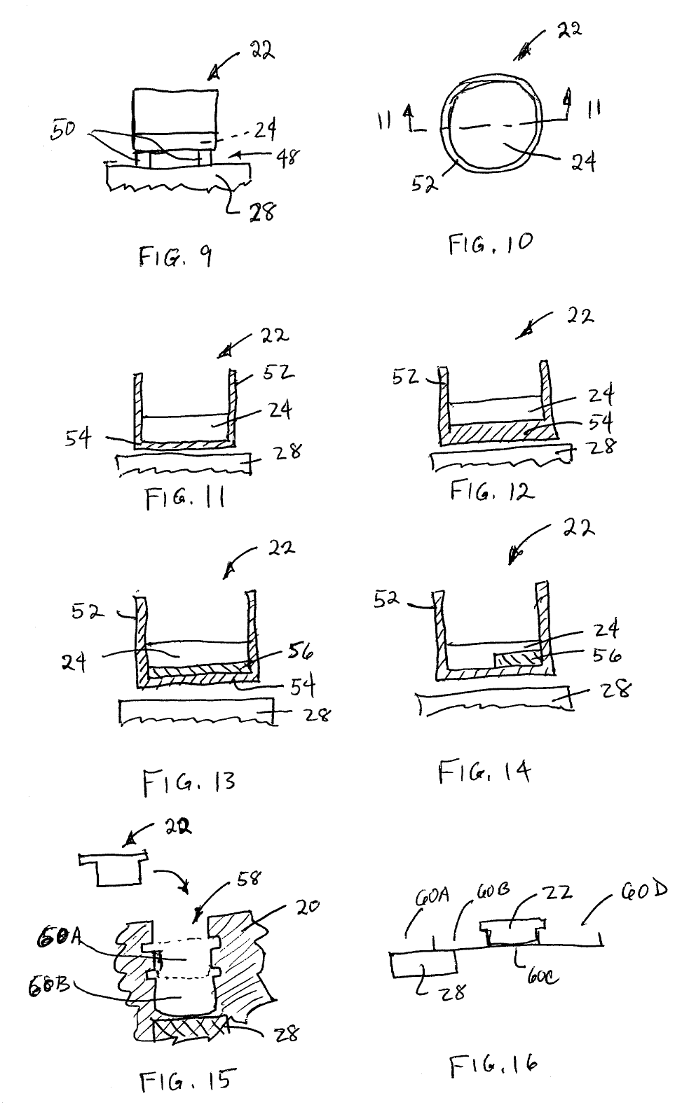

As shown in FIGS. 3-8, in other non-limiting embodiments, the method for controlling the emission of the volatile material(s) can be carried out by placing covers of different porosity between the volatile material(s) and the atmosphere. A container 22 containing volatile material 24 has a cover 44 positioned between the volatile material(s) and the atmosphere. The cover 44 can comprise any suitable material including, but not limited to a film. FIG. 4 shows an embodiment of a cover for the volatile material container shown in FIG. 3 which has relatively smaller sized pores that can be used with volatile materials having a relatively high volatility. FIG. 5 shows an embodiment of a cover for the volatile material container shown in FIG. 3 which has relatively larger sized pores that can be used with volatile materials having a lower volatility.

FIG. 6 shows an embodiment of an alternative cover for the volatile material container shown in FIG. 3. In the embodiment shown in FIG. 6, rather than comprising a material with a plurality of holes therein as shown in FIGS. 6-8, the cover can comprise a material that is inherently pervious. Such materials include, but are not limited to nonwoven materials. FIG. 7 is an enlarged view of the portion 46 of the cover 44 shown in FIG. 6, which comprises a material having a relatively low porosity that could be suitable for use with volatile materials having a relatively high volatility. FIG. 8 is an enlarged view of the portion 46 of the cover 44 shown in FIG. 6, which comprises a material having a relatively high porosity that could be suitable for use with volatile materials having a lower volatility.

FIG. 9 shows that in other non-limiting embodiments, the method can be carried out by providing a spacer 48 (or some other mechanism) that adjusts the distance between the volatile material 24 and a component that volatilizes or heats the volatile material such as a heater 28. In one embodiment, the spacer 48 can comprise one or more supports 50 that are disposed between the volatile material 24 and the heater 28. The supports 50 in such an embodiment may be separate components or part of another component. In one version of such an embodiment, the supports 50 may extend from a portion of the bottom surface of a fill or refill unit. Other embodiments are also possible.

FIGS. 10 and 11 are top and cross-sectional views of one embodiment of a fill or refill unit 22 containing volatile material 24. In the embodiment shown in FIGS. 10 and 11, the side walls 52 and the bottom 54 of the fill or refill unit 22 have about the same thickness. FIGS. 12-14 show that in other non-limiting embodiments, the method can be carried out by providing insulating material located between component that volatilizes or heats the volatile material such as a heater 28 to insulate different volatile materials to different degrees. As shown in FIG. 12, the insulating material can comprise the same material as the remainder of the container 22 holding the volatile material 24 which differs in thickness for different volatile materials. Alternatively, as shown in FIG. 13, the insulating material can comprise one or more materials 56 having different insulative values (for example, due to type or density of the insulating material). In a variation of these embodiments shown in FIG. 14, the extent of the coverage of the insulating material 56 can be varied. These embodiments, like the embodiments set forth above can adjust the energy applied to different volatile materials even if the device in which they are used has a heater that only has one mode, or a limited number of modes of operation.

FIGS. 15 and 16 show that in other embodiments, rather than providing supports or insulating material on the bottom surface of a fill or refill unit, the device that is used to emit volatile materials may comprise multiple positions or locations for the volatile material(s) that are different distances away from the component such as a heater which volatilizes the volatile material. In the embodiment shown in FIG. 15, the device comprises a receptacle 58 that comprises more than one insert positions, such as 60A and 60B for receiving a fill or refill unit 20 comprising volatile material. This type of configuration may be of interest in spacing the volatile materials different distances in a vertical direction from a heater 28. The embodiment shown in FIG. 16 also comprises multiple positions or receptacles (60A, B, C, and D) for the volatile material(s). This type of configuration may be of interest in spacing the volatile materials different distances in a horizontal direction from a heater 28.

FIGS. 17-21 show embodiments that comprise a volatile material, such as in liquid form 24, which utilize wicks 62 and optionally, but preferably, a heat source to volatilize the volatile liquid. Preferably, the unit is of a type that is capable of emitting the volatile material without a flame. In FIG. 18, a heating element 28 surrounds the wick. FIG. 18 shows two versions of an embodiment in which the effective emission energy of a single mode heating system can be changed by modifying the size (e.g., the diameter) of the wick 62. The smaller diameter wick 62A will be further from the heating element 28, and will also provide a lower surface area relative to the larger diameter wick 62B. This makes the smaller diameter wick 62A more suitable for more highly volatile materials, and the larger wick 62B more suitable for materials having lower volatilities.

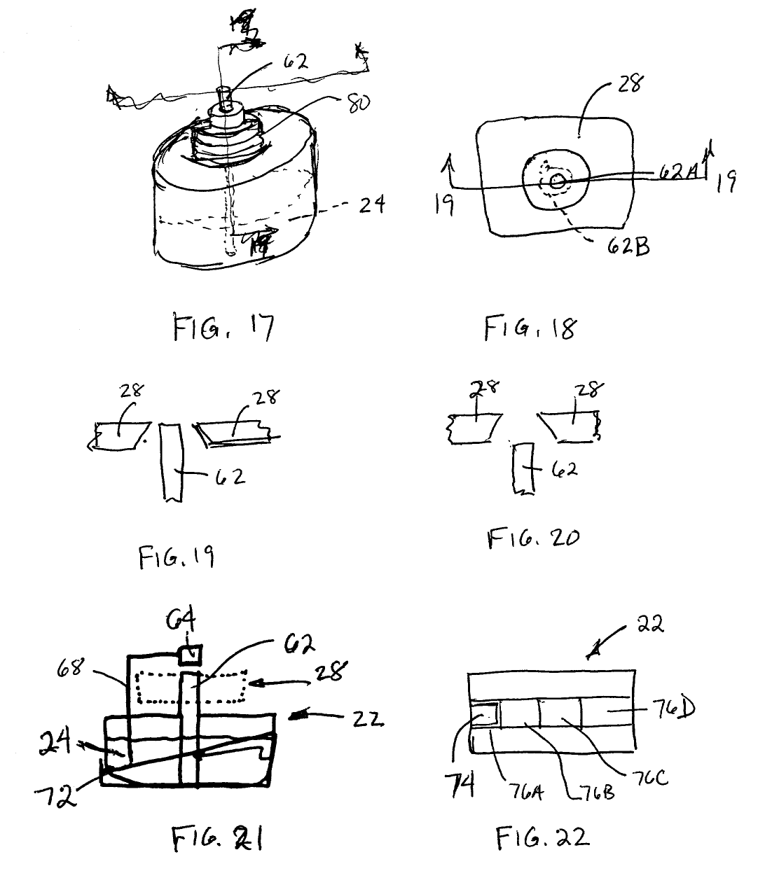

FIGS. 19 and 20 show alternative embodiments that utilize wicks and heating elements. In the embodiment shown in FIGS. 19 and 20, the heating elements 28 are tapered, however, non-tapered heating elements can also be used. In this embodiment, the position of the wicks 62 are varied relative to the heating elements 28 (with a longer wick 62 being used in the embodiment shown in FIG. 19, than in the embodiment shown in FIG. 20). The position of the wicks 62 can be varied in any suitable manner. In one variation of this embodiment, the wicks 62 can be of differing heights for different volatile materials. The height of the wicks 62 can be adjusted in any suitable manner, including but not limited to by screwing a fill or refill unit to different extents into a device containing the heater for different volatile material fill or refill units. One advantage to utilizing a tapered heating element 28 in such an embodiment is that it will provide a greater temperature increase per vertical distance that the wick 62 is inserted into the space between the heating elements 28, or portions thereof, than would straight-sided heating elements.

FIG. 21 shows another embodiment of a fill or refill unit 22 that utilizes a wick 62. A heating element 28 surrounds the wick 62 (though in other embodiments, it need not completely surround the wick 62). In this embodiment, an insulating material 64 is positioned in the region where the volatile material is first emitted (or in the “head space”). The insulating material 64 can create a cooler atmosphere in the region where the volatile material 24 is first emitted. This can cause some of the volatile material to condense, and thus, less of the volatile material to volatilize thereby reducing the rate of volatilization. In the embodiment shown in FIG. 21, the device comprises an adjusting component 66 that can be used to either establish or adjust the position of the insulating material 64. In this case, the insulating material 64 is suspended above the wick 62 by an element, such as a control arm 68. When the fill or refill unit is inserted into the device, the control arm 68 is in contact with an adjustment ramp 70. The fill or refill unit 22, or a portion thereof, can be rotated so that the lower end 72 of the control arm 68 can move up and down the ramp 70. This moves the control arm 68, and thus, the insulating material 64, up and down. By creating cooler and warmer conditions in the head space, condensation of the volatile material can be controlled, providing a consistent volatilization between systems (e.g., fill and refill containers) containing volatile materials with different volatilities. This type of arrangement can, like all of the other embodiments described herein, be used with other types of fill and refill units, and is not limited to use with containers comprising liquid volatile materials in jars with wicks.

FIG. 22 shows an embodiment in which the volatilization control component comprises a conductive element 74 that is part of a volatile material-containing article 22. The embodiment shown in FIG. 22 may comprise a fill or refill unit 22 for an article or device, and the unit may have a conductive element, such as a conductive label 74, that mates with contacts on and/or in the emitting article or device. In the embodiment shown, the conductive label can be located in one of four possible locations 76A, B, C, and D, each of which can contact one or more contacts on the emitting article or device. When the fill or refill unit 22 is associated with a device, the conductive label 74 can complete an electric circuit that powers a heating element in the device. The device has more than one circuit to power the heating element, each circuit yielding different heater outputs. The location of the conductive label 74 is such that only one of the circuits is completed when the fill or refill unit 22 is associated with the device. The position of the conductive label 74 can be varied depending on the volatile material contained in the fill or refill unit.

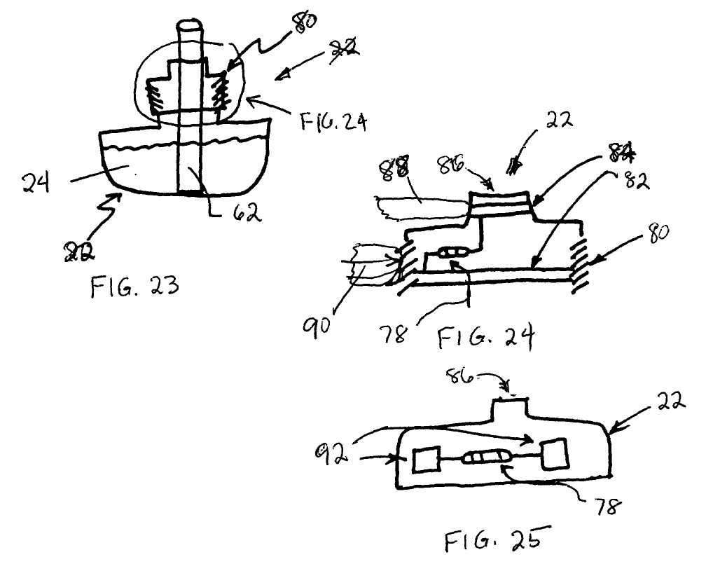

As shown in FIGS. 23-25, in other non-limiting embodiments, the volatilization control component 40 can comprise a resistor 78. The resistor 78 can, for example, provide higher resistance and thus send lower voltage to a heater and/or a fan in the case of materials that have higher volatility than it does for materials having lower volatility. If the volatilization control component is a resistor, it can comprise part of a device, or part of a volatile material-containing article (which includes initial fills and refills of the volatile material-containing article). A different resistor can be used with each different volatile material. FIGS. 23-25 show several different non-limiting configurations for how a resistor may be incorporated into an article or a device.

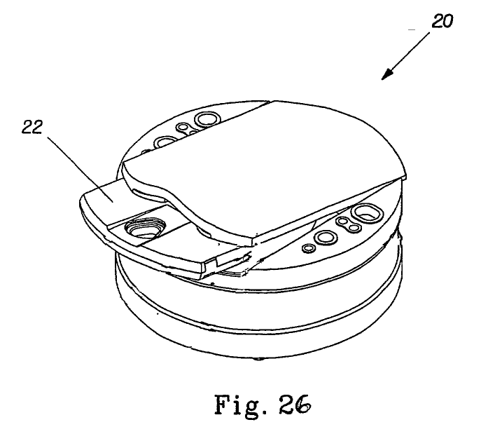

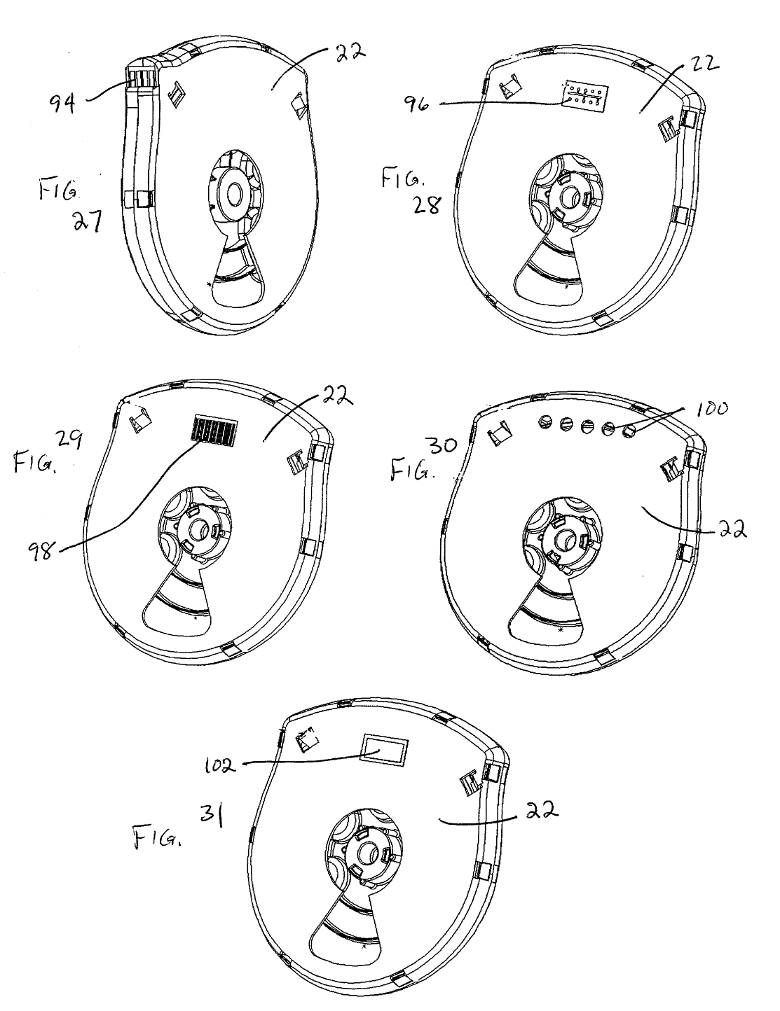

In these embodiments, the resistor 78 can be part of a fill or refill unit. The resistor 78 completes an electric circuit powering a heater and/or fan in the device when the fill or refill unit is associated with the device. In FIG. 23, the fill or refill unit 22 comprises a volatile material in a container, such as jar 22, with a wick 62 extending therefrom. On top of the unit is a threaded assembly 80 for attaching the unit 22 to a device. As shown in FIG. 24, the fill or refill unit 22 may further comprise a first metal contact ring 82 that in the embodiment shown, can be disposed in or around the threaded assembly 80, a second metal contact ring 84 that can be disposed adjacent to the mouth 86 of the jar, and the resistor 78. The fill or refill 22 containing the volatile material in the jar in such an embodiment can be screwed into the device. When the unit 22 is screwed into a device, the metal contact rings 82 and 84 can make contact with contacts 88 and 90 located on or in the device to complete an electic circuit that includes the resistor 78. FIG. 25 shows an alternative arrangement that utilizes a resistor 78. In FIG. 25, instead of having a threaded assembly for completing a circuit with a device, the fill or refill unit 22 containing the volatile material has point contacts 92 that can be aligned with contacts on the device.

FIG. 26 shows one non-limiting example of a relatively complex emission system in which the volatile material is inside a cartridge 22 that is inserted into a device 20. Such a system is capable of emitting multiple volatile materials, and is described in greater detail in PCT Patent Publication Nos. WO 02/09772, WO 02/09773, WO 02/09776, and their corresponding U.S. patent applications. FIGS. 27-31 show several non-limiting additional embodiments of mechanisms for communicating information to the device 20 that are located on the cartridges 22 for such a system. It should be understood, however, that these same mechanisms can be used on other, more simple emission systems than the system shown in FIG. 26.

The mechanisms that communicate information from the cartridge 22 to the device 20 may include, but are not limited to, the following: (1) electrical contacts 94 on or in the article 22 capable of being read by electrical circuitry (including, but not limited to a computer chip) in the device (FIG. 27); (2) conductive labeling 96 on or in the article 22 that mates with contacts associated with (e.g., in, on, or a part of) the device (FIG. 28), (3) optical mechanisms including, but not limited to bar coding 98 on the article 22 being read by the device (FIG. 29); (4) changes in topography on the article (such as raised portions, depressions, and holes 100 in the article 22) that are capable of being read by sensors in the device (FIG. 30); and (5) a radio frequency (RF) identification tag 102 on or in the article 22 which communicates with the device (FIG. 31).

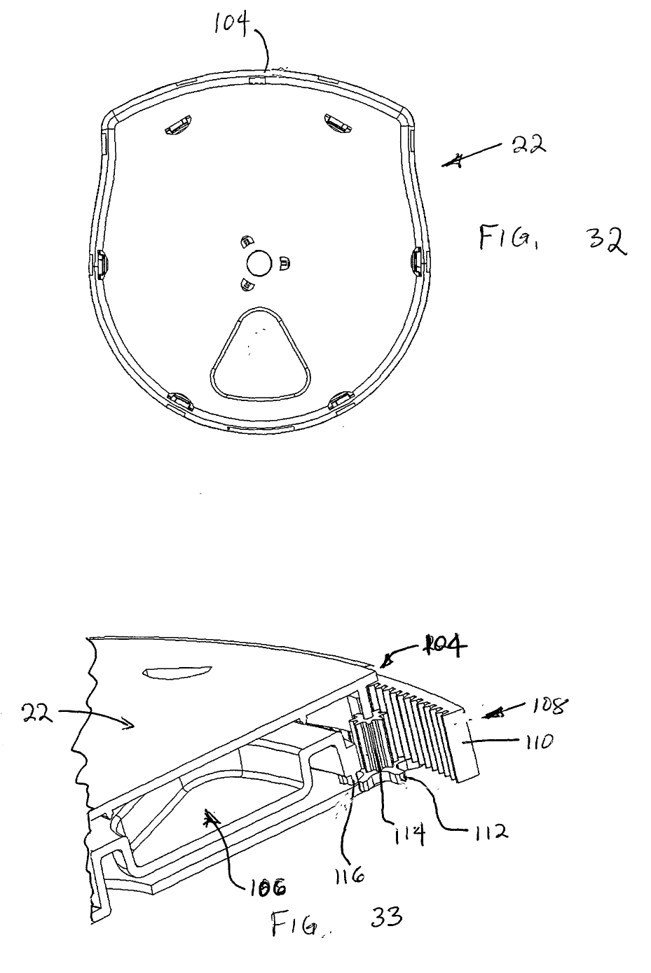

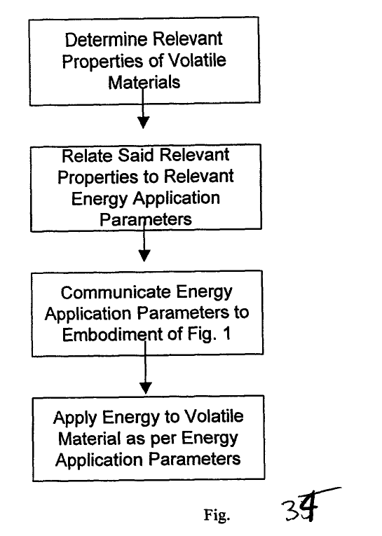

FIGS. 32 and 33 show one non-limiting example of an article capable of communicating information regarding the history of use of the article containing the volatile material(s). In the embodiment shown in FIGS. 32 and 33, the article comprises a cartridge 22 that has an indicator window 104 therein. Inside the cartridge 22 is a rotatable tray 106 containing pockets of volatile material. In the embodiment shown in FIG. 33, the cartridge 22 comprises a mechanism comprising an indicator designated generally by reference number 108, which may be in the form of a bar 110, and two gears 112 and 114. The rotatable tray 106 comprises an element such as a pin 116 that rotates the gears 112 and 114 and advances the indicator bar 110 each time the tray 106 rotates. If desired, the indicator bar 110 may have numbers and/or different color regions that are exposed when it is advanced to indicate the freshness of the cartridge. Other embodiments may use more sophisticated types of indicators can be used, including but not limited to indicators that keep track of the time period the volatile material(s) are emitted, and/or the intensity at which they have been emitted.

In certain embodiments, a system can be provided in which a fill or refill unit can modify the pulsing frequency of a pulsed heater and/or fan system. As set out above, a fill or refill unit can communicate a parameter (such as resistance, voltage, etc.) to a volatiation control component (such as a computer chip or integrated circuit) in a device.

In other embodiments, other types of components can be used to modify the pulse frequency of a pulsed system. In one non-limiting embodiment, both a device and a fill or refill unit can contain a resistor. For example, a timer circuit such as an LM 555 microchip from National Semiconductor, Texas, USA, can be part of the circuitry of the device. The microchip can be used in conjunction with a capacitor (located on the device or on the fill or refill unit) and the two resistors (one located on the device and one located on the fill or refill unit) so that the microchip functions as a multi-vibrator as described in the specification sheet for the microchip. One version of such an embodiment can be similar to the embodiments shown in FIGS. 23-25. The ratio of the resistances of the two resistors can be varied to precisely set the period (time between heating cycles) and the duty cycle of the vibrator. A wide range of resistance ratios can be effected by changing the value of the resistor in different fill or refill units. In other embodiments, the microchip can be eliminated, and a similarly-functioning circuit can be constructed using only capacitors, resistors, and optionally a relay.

In other embodiments, a simple electromechanical switch, much like the blinker for a car turn signal, can be used in series with a resistor supplied by a fill or refill unit. This switch will activate with a frequency that is directly proportional to the current running through it. According to Ohm's law, the current will be inversely proportional to the resistance in the circuit. Thus, by varying the value of the resistor in series with the switch, the current will be varied and, thus, the timing frequency of the switch can be varied. In other embodiments, the fill or refill unit can communicate a parameter (such as resistance, voltage, etc.) to a microcontroller, a microchip, or an integrated circuit, which controls the application of energy (including but not limited to the pulsing of a heater) to a volatile material.

More complex embodiments are possible. For example, in other embodiments, a system can be provided that comprises a device that has the ability to provide a number of different types of emission schemes. Numerous variations of such an embodiment are possible. In one variation of such an embodiment, the article containing the volatile material can be configured to indicate preferred volatilization energy application method, and there can be a mechanism on the device for the user to select the preferred volatilization energy application method. For example, in one embodiment, there can be an instruction on a volatile material-containing article the instructs the consumer to select a particular emission scheme when “playing” the volatile material-containing article. The instruction can be in any suitable form, including artwork and labeling. There can be any suitable level of consumer input in such a system. For example, in one version of such an embodiment in which the volatile material is scented material, the consumer can set one or more of the following: the desired intensity, duration, and room size. The device can interpret these settings based upon the particular scented material(s) that are in the article, and choose the proper emission scheme for the particular scented material(s). In another variation, there can be a label on the scent-containing article that instructs the user to set one or more of such parameters (desired intensity, duration, and room size), and also to select a particular emission scheme on the device for the particular article.

In another embodiment, a system can comprise a device with multiple volatilization energy application methods that are sequenced to run in a predetermined order. The article can contain multiple, separate volatile material(s), or units thereof, arranged in a fashion consistent with the predetermined sequence of volatilization energy application programs to provide optimal volatilization of each of the volatile material(s) or units. For example, a scent-containing article can communicate with a device mechanically and/or electrically to instruct the device that the scented material in a first location on (or in) the article should receive a first amount and/or duration of energy; and the scented material in a second location on (or in) the article should receive a different second amount and/or duration of energy, etc.

In another embodiment, a system can comprise a device with multiple volatilization energy application methods and the article comprises one or more separate volatile material(s), or units thereof. In this embodiment, the article is capable of communicating to the device which of the programs to activate for the one or various separate volatile material(s). A non-limiting example of this type of system is a device that is able to provide a fixed number of emission schemes (e.g., four emission schemes). When, for example, a scent-containing article is brought in for use with the device, the article can be configured to communicate which one of the four emission schemes the device should use when “playing” that article, or separately for the various scented materials contained therein.

In anoher embodiment, a system can comprise a device with a generalized volatilization energy application program and an article with one or more separate volatile material(s). In this embodiment, the article is capable of communicating to the device specific volatilization parameters for use by the generalized program for the one or each of the various separate volatile material(s). An example of such a system can include a device and an article in which the device is programed to obtain certain parameters from the article (e.g., parameters X, Y, and Z, where, for example, Z is the temperature). The parameter Z can be provided on or in the article (for example, the article can communicate to the device the temperature (e.g., 73.5° C.)) that should be imparted to a particular volatile material on or in the article. In a variation of such a system, the device is capable of delivering a range of volatilization energies and the device is capable of directly adjusting the intensity of volatilization energy application for the one or each of the various separate volatile material(s).

### Methods for Controlling the Release of Volatile Material(s)

There are numerous methods described herein for controlling the release of volatile materials. Some of these methods are directed to an overall process with begins with determining the relevant properties of the volatile material(s) and ends with release of the volatile material(s). Other methods described herein may only relate to processes of carrying out one or more steps in such an overall process (such as methods of commication between a volatile material containing article and an emission device). All of the methods described herein may comprise separate inventions. Any of the steps or embodiments of the methods described herein may be combined with any of the other steps or embodiments described herein to yield additional inventions. In some cases, portions of the methods, including any steps that occur during a portion of some of the methods described herein, may comprise separate inventions in their own right.

As depicted in FIG. 34, an overall method for controlling the release of volatile materials may be accomplished via the following steps: (1) determining the relevant properties of the volatile material(s); (2) relating the properties determined in step (1) to relevant energy application parameters; (3) communicating the energy application parameters to an emission device; and (4) applying energy to the volatile material(s) using the energy application parameters.

Examples of the manner of determining the relevant properties of the volatile materials are discussed in the preceding section of this specification (e.g., measurements of a material's volatility and other properties including, but not limited to molecular weight, flash point, vapor pressure, and thermo-gravimetric loss data). In more complex embodiments, the relevant properties may include, but are not limited to concentration required to deliver a desired result, psychophysical data such as odor detection limits, time required to become acclimated to a scented material (habituation), and time required for a scented material to lose its intensity or change its character. These properties may be determined by delivering a known concentration of the volatile material and measuring the result, such as extermination rate or intensity. As discussed in the preceding section, after determining the relevant properties of the volatile materials, the volatile materials may be classified into various different categories. The classification of volatile material(s) into groups with common, preferred volatilization parameters based on physical properties of the volatile material(s).

The next step in such an overall process is to relate the relevant properties to relevant energy application parameters. The energy application parameters may be varied in order to achieve a desired volatilization rate, intensity, etc. As discussed in the preceding section, energy application parameters may include, but are not limited to: temperature to which a volatile material is heated, the amount of time a volatile material is heated, exposed surface area of a volatile material, and airflow over a volatile material.

The next step in some embodiments of such an overall process is to communicate the energy application parameters to an emission device. In the most simple embodiments, communication of these parameters may be absent or unnecessary. For example, using different barrier materials to cover volatile materials with different volatilization properties allows different materials to be heated to the same temperature. No communication between the volatile material container and the emitter exists in this situation. Alternatively, a physical interaction may be used to effectively communicate a parameter to an emission device. In this case, the interaction may supersede the communication step and implement the energy application directly. This physical interaction could be, but is not limited to: a spacer built into the volatile container that places the volatile a specified distance from the heater thus achieving temperature control, a spacer that controls the position of a volatile container such that only portions of the volatile are exposed to the atmosphere, thus limiting evaporation. In more complicated embodiments, for example where different materials are heated to different temperatures, some means of communication may be required. This communication could occur in any of the manners set forth in the preceding section and includes, but is not limited to: binary communication from a microchip, binary communication from one or more mechanical switches that could be individually on or off, or analog communication of a value (such as a resistance).

One of the final steps in such an overall process is to apply energy to the volatile material(s) per the energy application parameters. In more complex embodiments, energy may be applied based on a computer program in which the communicated parameters are variables. As an example, a computer program may vary the application of energy to a volatile material by controlling the temperature and time that heat is applied to the volatile material, or the speed of airflow around a volatile material. The communication may then specify an exact heater temperature, or a range of temperatures, a duration of time to heat the volatile material, or an airflow rate. Additionally, other inputs may be factored into the program controlling the emission of the volatile material. For example, a user may wish to increase the relative concentration of a particular volatile material. This may be accomplished by selecting an intensity on a user interface. This intensity selection may then be read as a parameter in the emission program which would then vary an emission parameter based on this selection. Parameters which might be selected by a user interface may include but are not limited to: intensity of emission, total time of emission, and a sudden burst of emission. The user's input may modify the emission parameters, and will typically not completely override the volatile material specific input provided to the device.

Desired Output/Advantages

There are a myriad of benefits that may be associated with application of the methods described above. The methods, devices, articles, and systems described herein need not provide all or any certain number of these benefits. A partial listing related to the situation where the volatile materials are scented materials includes the following: (1) delivered scented material character and intensity are better maintained at a uniform level through the use of a given system; (2) intensities of scented materials can be normalized; (3) greater flexibility in selecting scented material material is afforded; (4) scented material habituation is greatly reduced; (5) more dynamic scented material experiences are made possible; (6) scented material is delivered efficiently, reducing cost and facilitating distinct scented material changes; and (7) easy-to-use, effective consumer controls are made possible.

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

While particular embodiments of the subject invention have been described, it will be obvious to those skilled in the art that various changes and modifications of the subject invention can be made without departing from the spirit and scope of the invention. In addition, while the present invention has been described in connection with certain embodiments thereof, it is to be undersood that this is by way of illustration and not by way of limitation and the scope of the invention is defined solely by the appended claims which should be construed as broadly as the prior art will permit.

What is claimed is:

1. An article containing at least one volatile material, said article comprising a mechanism that is capable of communicating information with a device, said device being of a type that enables the release of said at least one volatile material.

2. The article of claim 1 wherein said at least one volatile material comprises a scented material.

3. The article of claim 1 wherein said mechanism is capable of one of the following: transmitting information to a device; receiving information from a device; and both transmitting information to a device and receiving information from a device.

4. The article of claim 1 wherein said mechanism comprises at least one of the following: (1) an electrical contact associated with the article capable of being read by electrical circuitry associated with the device; (2) a conductive label associated with the article that mates with a contact associated with the device; (3) an optical mechanism associated with the article capable of being read by the device; (4) at least one change in topography on the article that is capable of being read by a sensor associated with the device; and (5) a radio frequency identification tag associated with the article which communicates with the device.

5. The article of claim 1 wherein said information is selected from the group comprising at least one of the following: (1) at least one volatile material-specific release or volatilization parameter, (2) volatilization energy application program selection, (3) a name associated with one or more volatile materials, (4) history of use of an article comprising volatile material, and (5) sequence of volatilization energy application programs.

6. The article of claim 1 wherein information relating to at least one volatilization parameter of at least one volatile material is stored on or in said article.

7. The article of claim 6 wherein said information is stored as a single parameter for more than one volatile material contained on or in said article.

8. The article of claim 5 wherein the information relating to the history of use is based on the application of at least one of the following volatilization parameters specific to at least one volatile material: the time period over which the volatile material is emitted, and the intensity at which the volatile material is emitted.

9. The article of claim 1 comprising two or more separate volatile materials, said article being for use in conjunction with a device that has more than one emission program for emitting volatile materials, wherein said article is capable of communicating to the device which emission program to activate for each of said two or more volatile materials.

10. The article of claim 1 comprising one or more separate volatile materials, said article being for use in conjunction with a device that has more than one emission program for emitting volatile materials, wherein said article is capable of communicating to the device specific volatilization parameters for each of one or more volatile materials.

11. An article containing at least one volatile material, said article of a type that is used in conjunction with a device for emitting volatile material, said article comprising an indicator which shows the amount that the article has been used, said indicator being changed by the device each time the article is used with the device.

12. A device for use in emitting volatile materials from an article containing a volatile material, said device comprising a component that is capable of communication with an article containing at least one volatile material.

13. A system for emitting volatile material, said system comprising:
(a) a device for use in emitting volatile materials from an article containing a volatile material; and
(b) an article containing at least one volatile material wherein said device and article comprise components of the system, and at least one of said article and said device comprises a mechanism that is capable of exchanging information with the other component of the system.

14. A method for controlling the emission of volatile material, said method comprising the steps of:
(a) providing a device for use by a user in emitting volatile materials from an article containing at least one volatile material;
(b) providing an article to a user, said article containing at least one volatile material, said article comprising a mechanism that is capable of exchanging information with said device; and
(c) associating the article with the device for the emission of at least one volatile material, wherein information is exchanged between the article and the device.

15. The method of claim 14 wherein said information is communicated in one of the following manners: from the article to the device; from the device to the article; and both from said article to said device and from said device to said article.

16. An article containing at least one volatile material, said article being configured for use in conjunction with a device for emitting volatile materials wherein the device provides at least two choices of volatile material-specific emission modes from which a user of the device may choose for emitting the volatile material from the article using the device, and said article comprises a mechanism that is capable of communicating information to a user of said article which choice of volatile material-specific mode of emission provided by the device to select.

17. The article of claim 16 wherein at least one volatile material comprises a scented material.

18. The article of claim 16 wherein at least two of the emission modes differ in at least one of the following parameters:
(a) temperature to which the volatile material is heated;
(b) duration of heating;
(c) intervals between heating;
(d) speed at which a fan that disperses the volatile material operates;
(e) duration of operation of a fan; and
(f) intervals between operation of a fan.

19. A device for use in emitting volatile materials from an article containing a volatile material, said device comprising at least two choices of volatile material-specific emission modes from which a user of the device may choose for emitting the volatile material from the article using the device.

20. A system for emitting volatile material, said system comprising:
(a) a device for use in emitting volatile materials from an article containing a volatile material, said device having more than one volatile material-specific mode of emission; and
(b) an article containing at least one volatile material, said article being configured for use in conjunction with said device wherein said article comprises a mechanism that is capable of communicating information to a user of said article to select a volatile material-specific mode of emission provided by the device.

21. A method for controlling the emission of volatile material, said method comprising the steps of:
(a) providing a device for use by a user in emitting volatile materials from an article containing at least one volatile material, said device having more than one volatile material-specific mode of emission;
(b) providing an article to a user, said article containing at least one volatile material, said article comprising a mechanism that is capable of communicating information to a user of said article to select a volatile material-specific mode of emission provided by the device; and
(c) communicating information from said article to the user of the article to select a volatile material-specific mode of emission provided by the device.

22. An article containing at least one volatile material, said article being configured for use in conjunction with a device for emitting volatile materials wherein said device provides more than one volatile material-specific mode for the release of volatile materials, said article comprising a mechanism that is capable of communicating information with at least one of the device or a person using the device to select a volatile material-specific mode of emission provided by the device.

23. A device for use in emitting volatile material from an article containing at least one volatile material, said device having more than one volatile material-specific mode for the release of volatile materials, said device comprising controls for controlling parameters relating to the release of a volatile material, said controls being related to factors that are independent of the specific type of volatile material.

24. A system for emitting volatile material, said system comprising:
(a) a device for use in emitting volatile material from an article containing at least one volatile material, said device having more than one volatile material-specific mode of emission, and controls for controlling parameters relating to the release of a volatile material, said controls being related to factors that are independent of the specific type of volatile material; and
(b) an article containing at least one volatile material wherein said article comprising a mechanism that is capable of exchanging information regarding the volatile material-specific mode of emission with at least one of said device or a person using the device.

25. A method for controlling the emission of volatile material, said method comprising the steps of:
(a) providing a device for use by a user in emitting volatile material from an article containing at least one volatile material, said device having more than one volatile material-specific mode of emission, and controls for controlling parameters relating to the release of a volatile material, said controls being related to factors that are independent of the specific type of volatile material;
(b) providing an article to a user, said article containing at least one volatile material, said article comprising a mechanism that is capable of communicating information to at least one of the user and the device regarding the volatile material-specific mode of emission; and
(c) allowing the user to operate said controls to control at least one factor that is independent of the specific type of volatile material.

26. The method of claim 25 wherein the controls operated by the user are capable of modifying the volatile material-specific mode of emission but not overriding said volatile material-specific mode of emission.

27. A method for releasing scented material, said method comprising:
providing at least one scented material; and
releasing said at least one scented material discontinuously and automatically so that said at least one scented material displays a non-constant in-air concentration over a period of time.

28. The method of claim 27 in which the scented material is released in at least one of the following manners: random bursts of scented material; the gradual increase or decrease in concentration through the duration of emission; and the intentional drop in concentration below sensory limits.

29. A system for emitting volatile material, said system comprising:
(a) a device for use in emitting volatile material from an article containing at least one volatile material, said device having more than one volatile material-specific mode of emission sequenced to run in a predetermined order; and
(b) an article comprising multiple, separate volatile materials in an arrangement that coincides with the predetermined sequence of volatile material-specific mode of emission on the device.

30. A system for emitting volatile material, said system comprising:
(a) a device for use in emitting volatile materials from an article containing at least one volatile material, said device being capable of delivering at least two different volatilization energy applications comprised of at least one of the following: volatilization energies, volatilization durations, and volatilization frequencies; and
(b) one or more articles each containing at least one volatile material,
wherein said device is capable of automatically adjusting at least one of the following for different volatile materials: the intensity, the duration, and the frequency of volatilization energy application, when said one or more articles are used with the device.

31. A system for emitting volatile materials, said system comprising:
(a) a device for use in emitting volatile materials from an article containing at least one volatile material, said device comprising an energy source, and being capable of providing volatilization energy to an article comprising one or more volatile materials; and
(b) an article containing one or more volatile materials for use with said device, wherein said article is configured to automatically alter the level of volatilization energy that reaches at least one of the volatile materials when said article is used in association with said device.

32. The system of claim 31 comprising at least one of the following: insulating material positioned between the energy source and the volatile material; films of different porosity positioned between the volatile material and the atmosphere; and a mechanism that adjusts the distance between the volatile material and the energy sourec.

33. A system for emitting volatile material, said system comprising:
(a) a device for use in emitting volatile materials from an article containing at least one volatile material, said device being capable of delivering one or more different volatilization energy application programs; and
(b) an article which is capable of altering the amount of energy received by the volatile material, dependent on the properties of the volatile material.
